(19) United States
(12) Patent Application Publication
(10) Pub. No.: US 2019/0170731 A1
(43) Pub. Date: Jun. 6, 2019

(54) METHOD TO MEASURE RELATIVE UTILIZATION OF AEROBIC GLYCOLYSIS BY POSITIONAL ISOTOPIC DISCRIMINATION

(71) Applicants: Da-Qing YANG, Austin, MN (US); Adrian HEGEMAN, Saint Paul, MN (US); Dana FREUND, Saint Paul, MN (US); Margot CLEARY, Austin, MN (US)

(72) Inventors: Da-Qing YANG, Austin, MN (US); Adrian HEGEMAN, Saint Paul, MN (US); Dana FREUND, Saint Paul, MN (US); Margot CLEARY, Austin, MN (US)

(21) Appl. No.: 16/308,950

(22) PCT Filed: Jun. 20, 2017
(86) PCT No.: PCT/US2017/038352

§ 371 Date: Dec. 11, 2018

Related U.S. Application Data
(60) Provisional application No. 62/352,165, filed on Jun. 20, 2016.

(51) Int. Cl. G01N 33/50; G01N 33/58

(52) U.S. Cl. CPC G01N 33/5038; G01N 33/58; G01N 2800/54; G01N 2800/52; G01N 2500/04

(57) ABSTRACT

Methods to detect aerobic glycolysis that employ isotopically labelled glucose are provided.

### CROSS-REFERENCE TO RELATED APPLICATIONS

This application claims the benefit of the filing date of U.S. application Ser. No. 62/352,165, filed on Jun. 20, 2016, the disclosure of which is incorporated by reference herein.

### GOVERNMENT SUPPORT

This invention was made with government support under 1R01CA157012-01A1, and IOS-1400818 and IOS-1238812, awarded by the National Institutes of Health and the National Science Foundation, respectively. The Government has certain rights in the invention.

### BACKGROUND

While normal cells produce ATP from glucose through oxidative phosphorylation, it is known that the majority of cancer cells also produce ATP by converting glucose to lactate even under aerobic conditions (DeBerardinis et al., 2008). The German Scientist, Otto Warburg discovered this phenomenon, termed aerobic glycolysis or the Warburg effect, nearly a century ago (Warburg, 1954). However, it was not appreciated until the development of the positron emission tomography (PET) scan technology. This imaging technology uses a radiolabeled glucose analog fluorodeoxyglucose (FDG) to detect metastatic lesions or assess treatment responses in patients with cancer by measuring elevated glucose uptake in vivo.

Results from PET scans have shown that dramatically increased glucose uptake is closely correlated with increased breast tumor aggressiveness and poor prognosis (Ueda et al., 2005). Evaluation of primary breast tumors using improved PET-computed tomography or PET/CT technology further indicates that higher levels of glucose uptake are significantly correlated with several biomarkers of breast cancer, such as negative status of estrogen receptor (ER) and progesterone receptor (PR), higher expression of erbB-2 (Her2), as well as tumor size and lymph node metastasis (Ueda et al., 2005).

Although PET or PET/CT scan results suggest that elevated glucose uptake may be one of the driving forces behind enhanced aerobic glycolysis in cancer cells, it is still unclear how closely the glucose uptake activity and glycolysis rate are correlated in cancer cells. In addition to glycolysis, lactate could also be produced from other metabolic pathways, such as the pentose phosphate pathway (PPP), during cancer cell metabolism (FIG. 1). Therefore, a method that can definitively measure the conversion of glucose to lactate through glycolysis in tumor cells is needed to accurately define the relationship between glucose uptake and glycolysis in cancer cells.

### SUMMARY

The ability of cancer cells to produce lactate through aerobic glycolysis is a consistent hallmark of cancer, including breast cancer. As described herein, a method employing positional isotopic labeling and mass spectrometry (MS). e.g., LC-MS, was established that can specifically measure the conversion of glucose to lactate through glycolysis. Using that method, it was shown that the rate of aerobic glycolysis is closely correlated with glucose uptake and lactate concentration in breast cancer cells. Significantly elevated production of [3-¹³C]lactate was also found in metastatic breast cancer cells and in early stage metastatic mammary tumors in mice, which may lead to the development of a biomarker for diagnosis of aggressive breast cancer.

The disclosure provides a method to detect aerobic glycolysis in a sample comprising cells. In one embodiment, the method detects glycolysis that is independent of (not associated with or not interfered by) PPP and/or glutaminolysis. The method includes providing a mixture comprising a sample obtained from cells, e.g., cancer cells, and labelled glucose. e.g., [1-¹³C]glucose, [1,2-¹³C2]glucose, [¹³C6]glucose, or 6,6-deuterium labelled glucose, measuring in the mixture the conversion of labelled glucose to labelled lactate, e.g., [1-¹³C]glucose to [3-¹³C]lactate, or deuterium labelled glucose to deuterium labeled lactate, over time using MS, and determining glucose uptake, lactate concentration or the rate of aerobic glycolysis in the cells in the sample, e.g., relative to control cells such as corresponding normal cells or corresponding cancer cells with low metastatic potential, or relative to t=0. In one embodiment the sample comprises pyruvate-free medium. In one embodiment, the sample is a physiological sample, e.g., a physiological fluid sample including but not limited to a blood sample, a plasma sample, a urine sample or a milk sample. In one embodiment, the sample is a tissue sample such as a tissue biopsy sample. In one embodiment, the cells comprise breast cancer cells. In one embodiment, the cells comprise prostate cancer cells, lung cancer cells, liver cancer cells, kidney cancer cells, ovarian cancer cells, bladder cancer cells, skin cancer cells, and the like. In one embodiment, the MS is LC-MS, which may be up to 1000 fold more sensitive than NMR and GC-MS. In one embodiment, glucose uptake in the cells in the sample over time is measured. For example, an increase in glucose uptake that is greater than 1.2-, 1.5-, 1.7- or 2-fold, or greater than control cells, or at t=0, is indicative that the cells in the sample have increased metastatic potential. In one embodiment, lactate concentration is measured. For example, an increase in lactate concentration that is 2%, 5%, 7%, 10% or greater than control cells, for instance, an increase from at least 0.025 mM to about 0.2 mM over time, is indicative that the cells in the sample have increased metastatic potential. In one embodiment, the relative rate of aerobic glycolysis is measured in vitro. For example, an increase in the relative glycolysis rate that is greater than 1.5-, 2-, or 3-fold, or greater than control cells, or at t=0, is indicative that the cells in the sample have increased metastatic potential.

Further provided is a method to detect the efficacy of a compound to alter aerobic glycolysis in cancer cells. The method includes contacting a compound, a sample comprising cells and an amount of labelled glucose, e.g., [1-¹³C]glucose, thereby providing a mixture; and measuring the conversion of labelled glucose to labelled lactate, e.g., [1-¹³C]glucose to [3-¹³C]lactate, in the mixture using mass spectrometry. In one embodiment, the cells are cancer cells. In one embodiment, the sample is a biopsy.

Also provided is a method to detect the effect of genetic mutation on aerobic

glycolysis in cancer cells. The method includes contacting cells, e.g., mammalian cells, having a mutation in a metabolic pathway; and measuring the conversion of [1-¹³C]glucose to [3-¹³C]lactate using mass spectrometry.

In one embodiment, a method to detect metastatic potential (pre-invasiveness) of cancer cells is provided. The method includes providing a mixture comprising mammalian cancer cells, e.g., human cancer cells, contacted with an amount of labelled glucose, e.g., [1-¹³C]glucose, [1,2-¹³C2]glucose, [¹³C6]glucose, or 6,6-deuterium labelled glucose. The conversion of labelled glucose to labelled lactate. e.g., [1-¹³C]glucose to [3-¹³C]lactate, in the mixture is measured using mass spectrometry and it is determined whether the cells have increased metastatic potential based on the presence or amount of the labelled lactate, e.g., [3-¹³C]lactate, or the rate of conversion of labelled glucose to labelled lactate, e.g., [1-¹³C]glucose to [3-¹³C]lactate, in the mixture. In one embodiment, the method is employed to detect pre-invasive breast cancer or other types of pre-invasive cancer cells, e.g., with the potential for metastatic invasiveness.

The disclosure also provides a method to detect aerobic glycolysis in vivo. The method includes collecting a physiological fluid. e.g., milk, blood or urine, or tissue sample from a mammal administered labelled glucose. e.g., [1-¹³C]glucose, [1,2-¹³C2]glucose. [¹³C6]glucose, or 6,6-deuterium labelled glucose, and measuring in the sample the ratio of [3-¹³C]lactate/unlabeled lactate, or deuterium labeled lactate/unlabeled lactate, using mass spectrometry. In one embodiment, the sample is a blood sample. In one embodiment, the sample is a milk sample. In one embodiment, the sample is a urine sample. In one embodiment, the sample is a tissue sample.

The disclosure provides a method to detect or diagnose pre-invasive or pre-malignant cancer in a mammal. The method includes collecting a physiological sample, e.g., a physiological fluid sample (for instance, a blood, milk or urine sample) or tissue sample, from a mammal administered labelled glucose, e.g., [1-¹³C]glucose. [1,2-¹³C2]glucose, [¹³C6]glucose, or 6,6-deuterium labelled glucose, and measuring in the sample the ratio of [3-¹³C]lactate/unlabeled lactate, or deuterium labeled lactate/unlabelled lactate, using mass spectrometry. In one embodiment, the ratio of [1-¹³C]lactate/unlabelled lactate, or the ratio of deuterium labelled lactate/unlabelled lactate, in the sample is measured using mass spectrometry. In one embodiment, a biopsy and [1-¹³C]glucose, or deuterium labeled glucose are mixed and the conversion of [1-¹³C]glucose to [3-¹³C]lactate, or the conversion of deuterium labeled glucose to deuterium labeled lactate, over time, e.g., the ratio of [3-¹³C]lactate/unlabeled lactate, or deuterium labeled lactate/unlabeled lactate, is measured using mass spectrometry. Samples having elevated levels of labelled lactate, for instance, relative to corresponding samples from a mammal that does not have cancer, are indicative of a mammal with a pre-invasive or pre-malignant cancer. In one embodiment, the sample is a physiological fluid sample. In one embodiment, the sample is a physiological tissue sample. For example, an increase in relative glycolysis rate or labelled lactate that is greater than 1.5-, 2-, or 3-fold, or greater than normal mammals, or at t=−0, is indicative that the mammal has pre-invasive or pre-malignant cancer.

In one embodiment, a method to monitor cancer recurrence in a mammal is provided. The method includes providing a mixture comprising a sample from the mammal comprising cells and an amount of ¹³C or deuterium labelled glucose; measuring in the mixture the conversion of the ¹³C or deuterium labelled glucose to ¹³C or deuterium labelled lactate, e.g., the ratio of [3-¹³C]lactate/unlabeled lactate or deuterium labeled lactate/unlabeled lactate, using LC-MS; and determining whether the mammal is at risk of cancer recurrence based on the presence or amount of the ¹³C or deuterium labelled lactate, or the rate of conversion of the ¹³C or deuterium labelled glucose to ¹³C or deuterium labelled lactate, e.g., the ratio of [3-¹³C]lactate/unlabeled lactate, or deuterium labeled lactate/unlabeled lactate, in the mixture. In one embodiment, the mammal is a human treated for breast cancer. In one embodiment, the mammal is a human treated for a cancer other than breast cancer. In one embodiment, the presence or amount of [3-¹³C]lactate, or the rate of conversion of [1-¹³C]glucose to [3-¹³C]lactate, e.g., the ratio of [3-¹³C]lactate/unlabeled lactate, or deuterium labeled lactate/unlabeled lactate, in the mixture is compared to the presence or amount of [3-¹³C]lactate, or the rate of conversion of [1-¹³C]glucose to [3-¹³C]lactate, in a control mixture or one or more samples from the mammal taken at an earlier point in time. In one embodiment, the presence or amount of deuterium labeled lactate, or the rate of conversion of deuterium labeled glucose to deuterium labeled lactate, in the mixture is compared to the presence or amount of deuterium labeled lactate, or the rate of conversion of deuterium labeled glucose to deuterium labeled lactate, in a control mixture or one or more samples from the mammal taken at an earlier point in time. In one embodiment, the sample is a physiological fluid sample. In one embodiment, the sample is a physiological tissue sample For example, an increase in relative glycolysis rate that is greater than 1.5-, 2-, or 3-fold, or greater than a control mammal, or at t=0, is indicative that the mammal has a recurrence of cancer.

In one embodiment, a method to monitor a therapeutic response to cancer therapy, e.g., chemotherapy, radiotherapy or immunotherapy, in a mammal having cancer is provided. In one embodiment, the method includes providing a mixture comprising a sample from the mammal comprising cells and an amount of ¹³C or deuterium labelled glucose; measuring in the mixture the conversion of the ¹³C or deuterium labelled glucose to ¹³C or deuterium labelled lactate, e.g., measuring the ratio of [3-¹³C]lactate/unlabeled lactate, or deuterium labeled lactate/unlabeled lactate, using LC-MS; and determining whether the mammal has a therapeutic response to the therapy based on the presence or amount of the ¹³C or deuterium labelled lactate, or the rate of conversion of the ¹³C or deuterium labelled glucose to ¹³C or deuterium labelled lactate, in the mixture. In one embodiment, the mammal is a human. In one embodiment, the mammal has breast cancer. In one embodiment, the mammal is a human with a cancer other than breast cancer. In one embodiment, the presence or amount of [3-¹³C]lactate, or the rate of conversion of [1-¹³C]glucose to [3-¹³C]lactate, in the mixture is compared to the presence or amount of [3-¹³C]lactate, or the rate of conversion of [1-¹³C]glucose to [3-¹³C]lactate, in a control mixture or one or more samples from the mammal taken at an earlier point in time. In one embodiment, the presence or amount of deuterium labelled lactate, or the rate of conversion of deuterium labeled glucose to deuterium labelled lactate, in the mixture is compared to the presence or amount of deuterium labelled lactate, or the rate of conversion of deuterium labeled glucose to deuterium labelled lactate, in a control mixture or one or more samples from the mammal taken at an earlier point in time. In one embodiment, the sample is a physiological fluid sample. In one embodiment, the sample is a physiological tissue sample. For example, an increase in relative glycolysis rate that is greater than 1.5-, 2-, or 3-fold, or greater than in a control mammal, or at t=0, is indicative that the mammal is not responding to therapy.

### BRIEF DESCRIPTION OF THE FIGURES

FIG. 1. A summary diagram showing the metabolism of [1-¹³C]glucose through the glycolysis and the pentose phosphate pathway. 100% glycolysis results in 1:1 ¹³C to ¹²C at C3 of lactate, but all of the labeled carbon will be lost as ¹³CO₂ if the glucose is metabolized via the pentose phosphate pathway.

FIG. 2. MDA-MB-231 cells exhibit higher glucose uptake than MDA-MB-453 cells. Sub-confluent cells were serum-starved overnight. Cells were then washed with PBS and cell culture medium was replaced with glucose- and serum-free medium. Fluorescently-tagged 2-NBDG (Cayman Chemical) was then added at a concentration of 30 μg/mL for 30 minutes. After addition of 2-NBDG, cells were treated with 100 nM insulin for another 45 minutes. Glucose uptake was then measured as described hereinbelow. The graph represents the averages of 2-NBDG glucose uptake ±SEM from 3 individual experiments (p<0.05).

FIG. 3. MDA-MB-231 cells exhibits higher rate of glycolysis than MDA-MB-453 cells. Equal number of MDA-MB-231 and MDA-MB-453 cells were cultured in DMEM medium containing 10% FBS. Sub-confluent (60-80% confluency) cells were serum starved overnight. Cells were then washed with PBS and cell culture medium was replaced with glucose/pyruvate/serum-free medium. The labeling of D-[1-¹³C]glucose (10 mM) was initiated following 90 minutes of glucose/pyruvate starvation. 40 μL of cell culture medium was taken at indicated time points and later diluted with 160 μL of methanol to precipitate the proteins. The LC-MS analysis of the cell culture medium was performed on a Q-Executive mass spectrometer. The graph represents the averages of glycolysis rates ±SEM from 3 replicates.

FIG. 4. The relative rates of aerobic glycolysis in MDA-MB-231 and MDA-MB-453 cells are correlated with the lactate production. The cell culture medium obtained from the [1-¹³C]glucose labeling experiments performed in FIG. 3 was subjected to lactate concentration assay. Lactate was measured using an L-Lactate Assay Kit following the protocol from the manufacturer. The graph represents the averages of lactate concentrations ±SEM from 3 individual experiments.

FIG. 5. Mice with early stage metastatic mammary tumors display significantly elevated rate of glycolysis in serum samples. A) C57BL/6 mice were either injected orthotopically in the fourth inguinal mammary fat pad with E0771 cells in the saline or injected with saline only. After 3-4 weeks, when the tumors became visible, mice with or without mammary tumors were fasted overnight and injected via tail vein with 0.2 mL of IM sterile [1-¹³C]glucose the next morning. Blood was drawn via facial vein one hour after injection. Blood samples were later centrifuged, and mouse serum was collected and processed for LC-MS analysis. The results are presented as [1-¹³C]lactate/unlabeled lactate in serum samples from mice (n=6) with early stage metastatic mammary tumors versus mice (n=6) without tumors (p<0.05). B) The serum samples obtained from the mouse experiments performed above were subjected to lactate concentration assay. Lactate was measured using an L-Lactate Assay Kit following the manufacturer's instructions. The graph represents the averages of lactate concentrations ±SEM from 3 individual experiments.

FIG. 6. A re-presentation of the isotopic labeling results in cultured breast cancer cells from FIG. 3. The results show the relative flux of [1-¹³C]glucose through glycolysis versus pentose phosphate pathway in its conversion to lactate, after three hours of labeling the breast cancer cell lines with [1-¹³C]glucose.

FIGS. 7A-B. A) Sub-confluent MDA-MB-231 cells were serum-starved overnight. Cells were washed with PBS and were then pre-treated with 10 μM KU-55933 (Halaby et al., 2008) for 30 minutes in the glucose- and serum-free medium. Fluorescently-tagged 2-NBDG (30 μg/ml) was then added for 30 minutes. Cells were treated with 100 nM insulin for another 45 minutes. Glucose Uptake was then measured following the manufacturer's instructions (Cayman Chemical). B) MDA-MB-231 cells were cultured in DMEM containing 10% FBS. After reaching −80% confluent, cells were serum-starved overnight. Cells were then washed with PBS and incubated in serum- and glucose/pyruvate-free DMEM for 90 minutes. The labeling was initiated after replacing the medium with fresh serum- and glucose/pyruvate-free DMEM supplemented with 10 mM D-[1-¹³C]-glucose ±10 μM KU-55933. After 9 hours of incubation. 40 μL of medium was taken and diluted with 160 μL of methanol to precipitate the proteins. For LC-MS analysis, 2 μL of the supernatant were injected and analyzed with the Q-Exactive mass spec. The bar graph represents the average relative glycolysis rate ±SEM from 3 individual experiments (*p<0.05). Samples were also taken at 1, 3, and 6 hours of labeling, which show significant inhibition of glycolysis rate by KU-55933 as well.

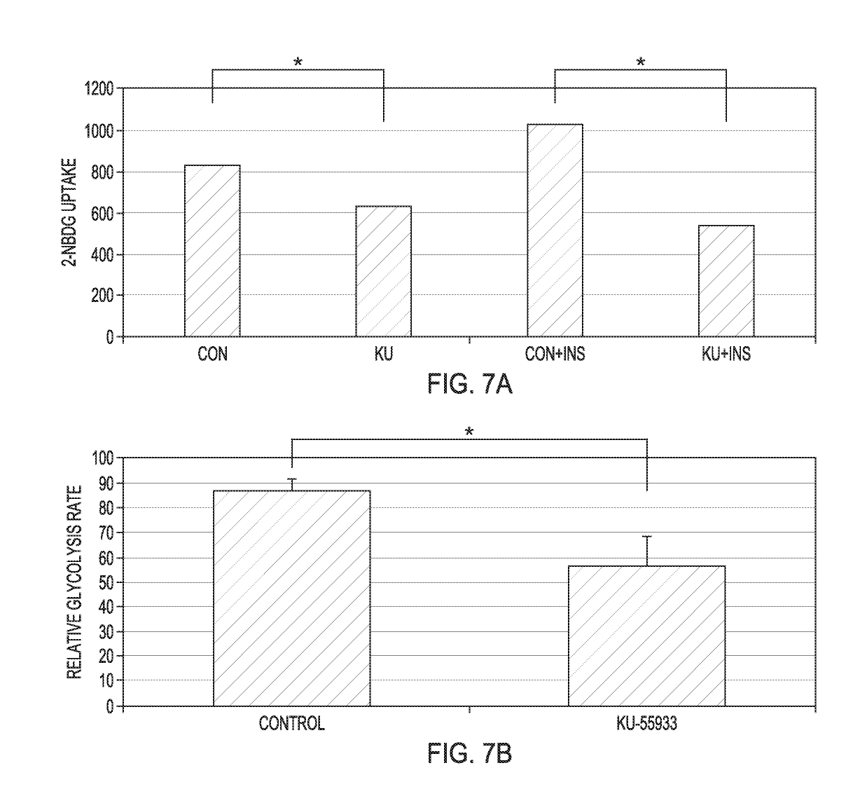

### DETAILED DESCRIPTION

Metabolomics is a field that encompasses a variety of analytical approaches that are unified with the common goal of high-throughput measurement of small molecules or metabolites found within cells and biological systems (Hegeman, 2010). Among these different analytical approaches, stable isotopic labeling or tracing is an effective method for determining the relative contribution of a substrate to a particular metabolic pathway, and when coupled to mass spectrometry (MS), which enables quantification of the relative abundance of molecules with different isotopic compositions.

The present disclosure describes a positional isotopic labeling and a mass spectrometry-based method, e.g., liquid chromatography (LC)-MS-based method, that can specifically measure the conversion from glucose to lactate through glycolysis in cancer cells. The rate of aerobic glycolysis obtained by this method was shown to be closely correlated with glucose uptake activity and lactate concentration in breast cancer cells. The results further indicate significantly elevated production of [3-¹³C]lactate in metastatic breast cancer cells and in early stage metastatic mammary tumors in mice, which may lead to the development of a promising biomarker for invasive breast cancer.

The detection method can be used to measure elevated production of [3-¹³C]-lactate in serum samples as a biomarker for pre-invasive breast cancer following the injection of a small amount of stable isotope-labeled [1-¹³C]-glucose into patients after overnight fasting. This is a minimally invasive, non-radioactive, and economic procedure that can be performed in women who have already had DCIS, detected by mammography screenings and/or MRI. This method can also be used to monitor the therapeutic response and/or tumor relapse in patients treated with chemotherapeutic agents against glycolysis. In one embodiment, the method may be employed for high-throughput screening of drugs that can specifically inhibit aerobic glycolysis in various types of cancer cells. The method can also be used for biomedical research detecting the effects of different pathophysiological conditions or genetic mutations on aerobic glycolysis in cancer cells, which may aid in the development of personalized therapy for cancer patients.

In contrast to earlier methods, including measurement of acidity in the cell culture medium (Seahorse Biosciences) or detection of lactate by an enzyme-based approach (various biotech companies), the present method can measure relative production of lactate from a single metabolic pathway, rather than multiple metabolic pathways.

Compared to the earlier methods, the present method is more sensitive. It can accurately trace the conversion from glucose to lactate through glycolysis in cultured cells or in vivo in animal models of cancer, since it measures the conversion from [1-¹³C]-glucose to [3-¹³C]-lactate without the interference of the pentose phosphate pathway (the stable ¹³C entering the pathway becomes CO₂) and gluaminolysis (no labeled glutamine added into the medium or injected into the body). It can also be used to assess the efficacy of anti-glycolysis drugs in vitro and in vivo. In addition the method can be used in high-throughput screening of drugs that are capable of inhibiting aerobic glycolysis in cancer.

The invention will be further described by the following non-limiting example.

### EXAMPLE

### Materials and Methods

### Materials.

Glucose and lactate were purchased from Sigma. [1-¹³C]glucose and [3-¹³C]lactate were purchased from Cambridge Isotope Laboratories.

### 2-NBDG Uptake Assay.

Glucose uptake was analyzed using a 2-NBDG (2-deoxy-2-[(7-nitro-2,1,3-benzoxadiazol-4-yl)amino]-D-glucose, a fluorescently labeled 2-deoxyglucose) glucose uptake kit from Cayman Chemical. Briefly, cells were plated at 200,000 cells/well in a 24-well plate and allowed to grow to sub-confluency. Cells were then serum starved overnight. The next morning, cells were incubated in serum- and glucose-free medium for 30 minutes. Cells were then incubated with 30 μg/mL 2-NBDG for another 30 minutes. After incubation, cells were treated with 100 nM insulin for 45 minutes. Cells were then transferred to a clear-bottom black 96 cell plate. The plate was subjected to centrifugation at 400×g for 5 minutes. The medium was removed by aspiration and cells were washed with PBS before the addition of cell-based assay buffer (provided in the kit) to each well. Signal intensity was measured with a Synergy 2 (BioTek) microplate reader at excitation/emission=485/535 nm.

### Lactate Concentration Assay.

Lactate was measured using a L-Lactate Assay Kit (Eton Biosciences) following the protocol from the manufacturer. Briefly, samples were diluted 1:10 with nano pure water to 50 μL total volume and then mixed with 50 μL of L-lactate assay solution provided in the kit in a 96-well plate. The plate was then incubated at 37° C. for 30 minutes. The absorbance was measured at a wavelength of 492 nm with a Multiskan Ascent (Labsystems) microplate reader.

### Cell Culture and 1-¹³C-Glucose Labeling.

MDA-MB-231 is an aggressive breast cancer cell line that has strong invasive capability, whereas MDA-MB-453 is a breast cancer cell line that displays relatively low or non-invasive capability (Zhang et al., 2013; Wang et al., 2011). These breast cancer cells were grown in DMEM supplemented with antibiotics and 10% fetal bovine serum. Before the labeling experiment, equal cell numbers (5×10⁵/well) were plated on a 6-well plate and allowed to grow to subconfluency. The labeling procedure was modified from one described in Ben-Sahra et al. (2013). Briefly, cells were serum-starved overnight. The next morning, cells were washed with PBS and cell culture medium was replaced with serum/glucose/pyruvate-free medium for 90 minutes. Following glucose/pyruvate starvation, the medium was replaced with fresh serum/glucose/pyruvate-free medium supplemented with 10 mM [1-¹³C]glucose to initiate isotopic labeling, and cell culture medium (40 μL) was taken at 1 hour. 3 hour. 6 hour and 9 hour time points for further LC-MS analysis.

### Animal Study

C57BL/6 female mice (Harlan) at 12 weeks of age were injected orthotopically in the fourth inguinal fat pad with about 200,000 syngeneic E0771 cells in saline or injected with saline only at the same site. E0771 is a mouse mammary tumor cell line derived from C57BL/6 mice and is metastatic in vivo when inoculated in C57BL/6 mice (Chen et al., 2012). After 3-4 weeks, mice with or without mammary tumors were fasted overnight, and then the next morning, 0.2 mL of IM sterile [1-¹³C]glucose was infused into each mouse by tail vein injection. At this early stage of the tumor development, mouse body weight (average=about 23 g/mouse) did not exhibit significant changes between the control and the tumor bearing group. One hour following injection, blood was collected from the mice. Mouse serum was prepared following centrifugation and was stored at −80° C. for further LC-MS analysis. Mice were later sacrificed and mouse tumors and mouse tissue samples were collected for further pathological analysis to confirm tumor grades and metastasis.

### LC-MS Analysis.

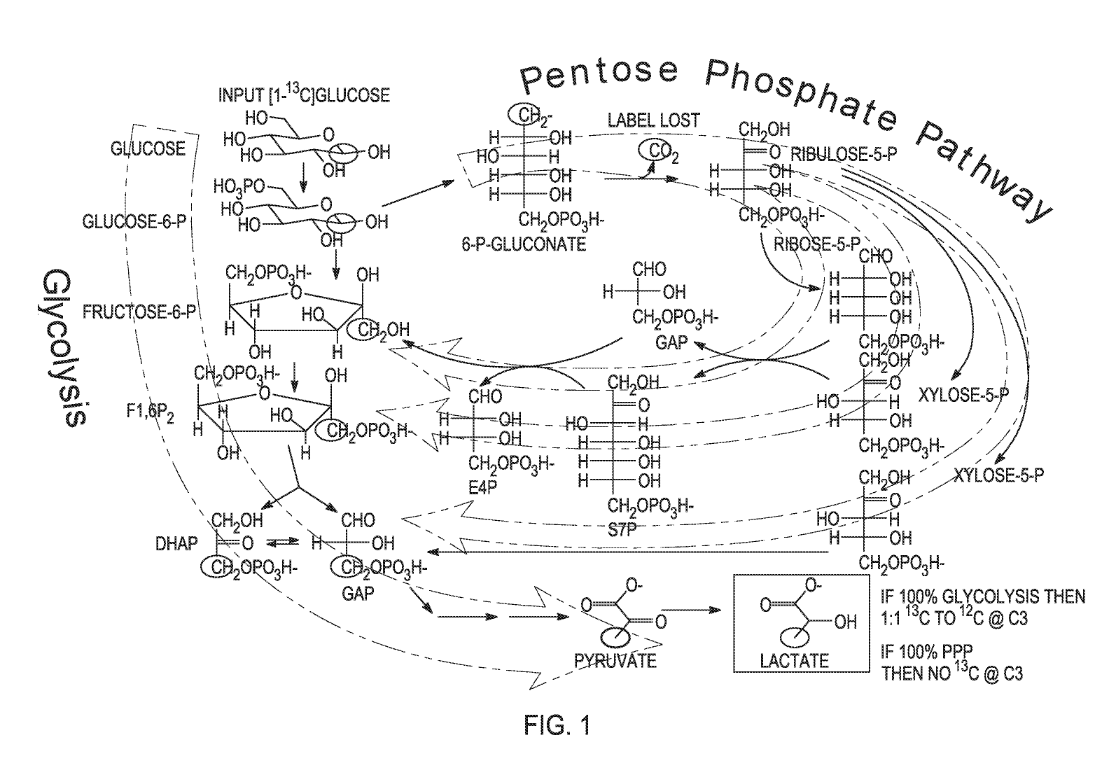

Cell culture medium taken, or mouse serum prepared from the cell and mouse isotopic labeling experiments, was mixed with 100% methanol at 2:8 (40 μL/160 μL) ratio to precipitate the proteins. After continuous mixing by vortex for 10 minutes, the mixtures were subjected to centrifugation for 10 minutes at 13,000×g and the supernatant was used for LC-MS analysis. Briefly, 2 μL of the supernatant from each sample was injected into a ZIC-HILIC column. 100 mm×2.1 mm. 3 μm from Merck SeQuant (Darmstadt, Germany) using an Ultimate 3000 UHPLC system coupled to a Q Exactive Quadrupole-Orbitrap hybrid mass spectrometer (Dionex/Thermo Fisher Scientific, Bremen, Germany) with a heated electrospray ionization (HESI) source. An eight-minute gradient using a flow rate of 400 μL/minute with mobile phase A (0.1% formic acid in water) and B (0.1% formic acid in acetonitrile) with the following gradient: initial. 98% B: 0-6 minute. 98-40% B; 6-8 minute 40% B. The following MS conditions were used: full scan mode in negative, scan range of 80-1200 m/z, a resolution of 35,000 (at m/z 200), target automatic gain control (AGC) of 1×10⁶, and maximum fill times of 200 ms. Data were collected and viewed in Xcalibur software version 2.2 (Thermo Scientific, Bremen, Germany). The identity of lactate was verified by retention time, accurate mass, and fragmentation spectra using an authentic standard. The raw files were converted to mzXML files with msConvert tool from ProteoWizard (Chambers et al., 2012). Both XCMS and ProteinTurnover software packages implemented in R were used for data processing (Smith et al., 2006). An example of the code used for data processing can be found here https://github.com/dfreund/Lactate1-13C.git. The relative rate of glycolysis for each cancer cell line was measured by the incorporation of [1-¹³C]glucose into [3-¹³C]lactate. Briefly, extracted ion chromatograms (EICs) for a specific retention time window were generated for the lactate isotopomers: [M₀]=89.024 m/z (unlabeled lactate) and [M₁]=90.028 (labeled [3-13C]lactate). A retention time correlation strategy is used, the EICs for each point are plotted, and linear regression is performed on the plot. The slope of the line is the ratio of intensity for the isotopomers (M₁/M₀, [3-13C]lactate/unlabeled lactate). The relative flux of glucose into lactate through the glycolysis pathway and the pentose phosphate pathway (PPP) was calculated using the ratios of labeled [3-13C]lactate (from glycolysis) versus [unlabeled lactate+labeled [3-¹³C]lactate] (from both the glycolysis and the PPP pathway) during the initial labeling phase, after depleting residual lactate in the medium by glucose/pyruvate starvation followed by a change of old medium with new medium containing [1-¹³C]glucose. Specifically, the following equation is used to calculate the percentage of glycolysis: 2*(M₁/(M₀+M₁)*100%. An amplification factor of 2 was used to reflect the isomerization or isotope exchange between DHAP and glyceraldehyde 3-P in the glycolysis pathway (FIG. 1).

### Results

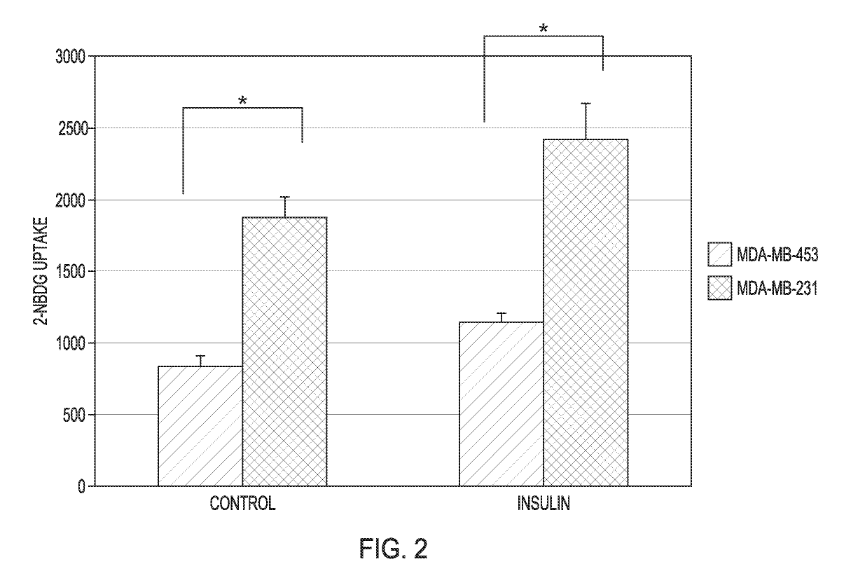

Glucose uptake activity of two breast cancer cell lines, MDA-MB-231 and MDA-MB-453, was measured by the 2-deoxy-glucose incorporation method using a fluorescently-tagged 2-deoxyglucose, 2-NBDG. The results show that both cell lines exhibit enhanced glucose uptake in response to insulin stimulation. Interestingly, it was found that MDA-MB-231, an aggressive metastatic breast cancer cell line, exhibits much greater (about 2 fold) glucose uptake activity under both basal and insulin-mediated conditions than that of MDA-MB-453, a breast cancer cell line with low metastatic capability (FIG. 2) (Zhang et al., Wang et al., 2011). To directly determine the link between glucose uptake and glycolysis in cancer cells, a stable isotopic labeling and LC-MS-based method was established to measure the conversion of [1-¹³C]glucose to [3-¹³C]lactate through glycolysis in cancer cells. This LC-MS method was developed for rapid separation and detection of lactate in 80% methanol extracts from medium or serum samples. Identification of lactate was confirmed with an authentic standard, verifying retention time, accurate mass, and the fragmentation or tandem mass spectra (MS/MS) (data not shown).

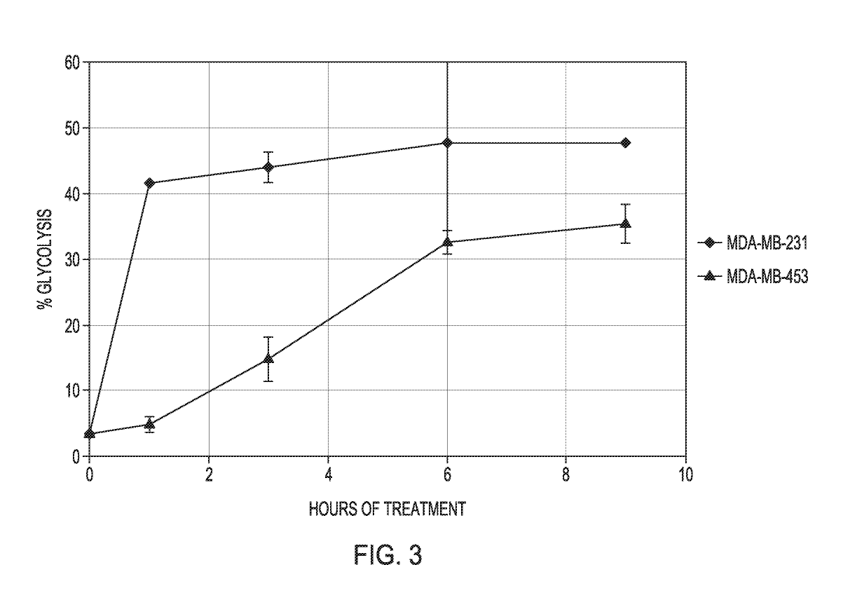

Using this method, the lactate production from glucose was measured in MDA-MB-231 and MDA-MB-453 cells. Consistent with enhanced glucose uptake in breast cancer cells, the results indicate production of [3-¹³C]lactate from [1-¹³C]glucose in these breast cancer cells, even under normal aerobic growth conditions. Interestingly, it was found that MDA-MB-231 cells exhibit dramatically increased production of [3-¹³C]lactate from [1-¹³C]glucose as compared to MDA-MB-453 cells (FIG. 3). Lactate production was compared in MDA-MB-231 cells versus other non- or low-metastatic breast cancer cell lines and it was found that MDA-MB-231 cells also exhibit higher production of [3-¹³C]lactate than those cell lines (data not shown).

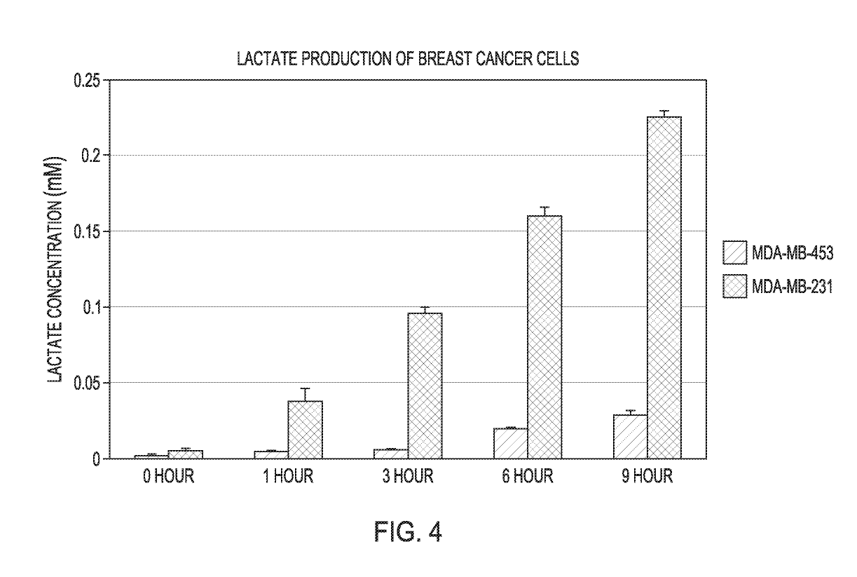

Initially, it was thought that lactic acid/lactate was a waste product of glycolysis, but it is now known that elevated levels of lactate are closely correlated to increased tumor aggressiveness and poor prognosis (Doherty and Cleveland, 2013; Dhup et al., 2012). To determine whether the results from LC-MS method agree with the amount of lactate in the medium that was secreted from the cancer cells, the lactate concentrations in the cell culture medium were measured using a commercially available spectrophotometric lactate assay kit. The results indicate that the measurements (FIG. 4) agree with the aerobic glycolysis rates obtained using the LC-MS method.

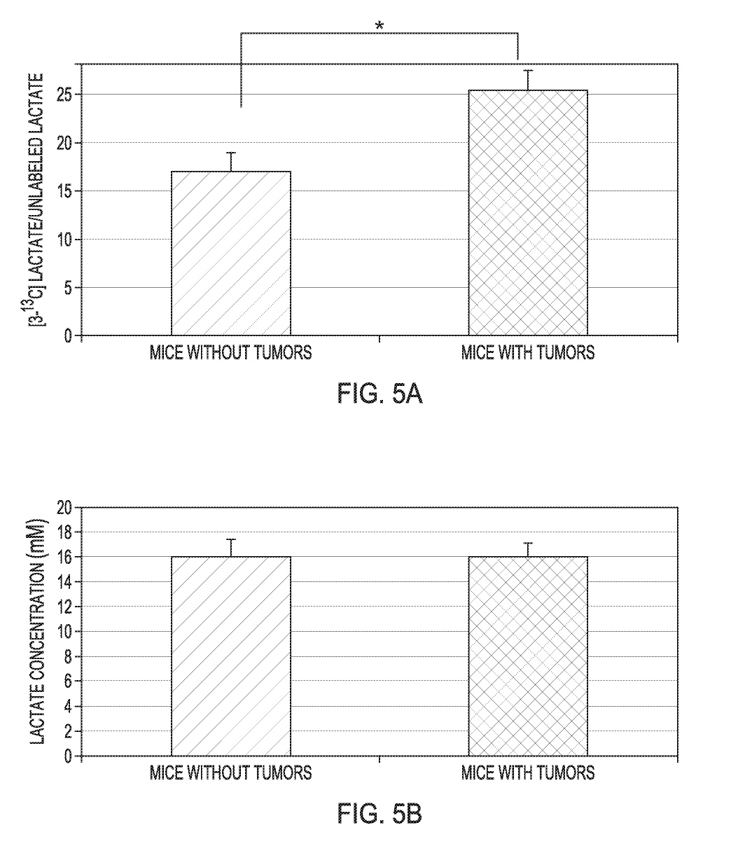

Next, lactate production rate was compared in C57BL/6 mice with or without mammary tumors. C57BL/6 mice were either inoculated with E0771 cells, a metastatic mouse mammary tumor cell line derived from the same mouse species (Chen et al., 2012), or inoculated with saline. After tumors derived from E0771 cells became visible, the lactate production rates in these mice were monitored following overnight fasting of the mice. A significant elevation of [3-¹³C]lactate was observed in the serum samples from mice bearing early stage metastatic mammary tumors compared to those from mice bearing no mammary tumors (FIG. 5A).

In contrast to cultured cancer cells in which lactate is produced by single-batch of uniformed cells under well-controlled growth conditions, lactate production in mice also involves lactate produced by other organs, namely the muscle tissue. Therefore, basal levels of lactate concentration were measured in serum samples from C57BL/6 mice with or without mammary tumors. Interestingly, it was observed the same level of lactate concentration between mice with or without mammary tumors (FIG. 5B). These results suggest that the LC-MS method for monitoring transient lactate incorporation rates is very sensitive in differentiating the lactate production in mice with or without metastatic tumors, despite the same basal levels of lactate in these mice.

### Discussion

The ability of cancer cells to produce large amounts of lactate through aerobic glycolysis is coupled to high rates of glucose uptake (Chen and Russo, 2012). In fact, increased glucose uptake and glycolysis are among the most consistent hallmarks of cancer, including breast cancer (DeBerardinis et al., 2008; Chen and Russo, 2010). These alterations in cellular metabolism play key roles in protecting cancer cells from apoptosis by rendering them independent of the need for growth factors and other environmental stimuli. Magnetic Resonance Spectroscopy (MRS), also called NMR spectroscopy, has been primarily used to detect elevated glycolysis or lactate production from glucose as an indicator of tumor development in brain cancers such as glioma (Schupp et al., 1993). However, the usage of this method in other types of cancer has been limited by the sensitivity of the traditional NMR technique (Wolfender et al., 2014).

However, recent advancements in LC-MS have significantly improved the sensitivity of this method compared to traditional GC-MS- or NMR-based technologies (Wolfender et al., 2014), which make it feasible to detect very low concentrations of small molecules or metabolites. Furthermore, one of the most common methods for metabolic tracing of glucose metabolism is to use [2-¹³C]glucose, but it is difficult to distinguish between different pathways leading to production of lactate using this isotopic labeled glucose molecule. Moreover, there are no other currently available detection methods that can monitor the production of lactate from glucose through glycolysis in cancer cells that are not interfered with by other metabolic pathways. The commercially available methods, including measurement of acidity in the cell culture medium (Seahorse Biosciences) or detection of lactate by an enzyme-based approach (various biotech companies), only measure concentrations of lactate, the end product of glycolysis, which could be from multiple metabolic pathways.

In contrast to these methods, the method described herein in cultured cancer cells is not only much more sensitive, but it can also accurately trace, at least in the initial labeling phase, the conversion of glucose to lactate through glycolysis without the interference of other pathways such as the PPP pathway and glutaminolysis. As shown in FIG. 1, the carbon at C1 of glucose (anomeric carbon) becomes CO₂ in the PPP pathway. In addition, no labeled glutamine was added into the medium or injected into the mice so lactate production from glutaminolysis is not traced. Indeed, the present results show a dramatically enhanced production of [3-¹³C]lactate from [1-¹³C]glucose in cancer cells, which agrees with the enhanced glucose uptake activity in breast cancer cells and the aggressiveness of mouse mammary tumors.

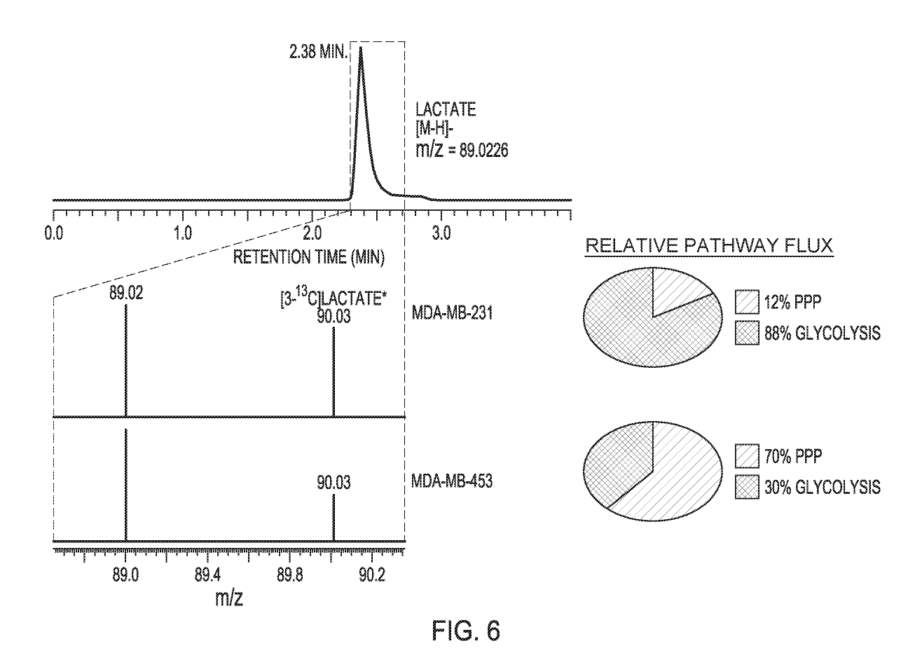

The detection method established in this study has shown promising results comparing the glycolysis rates in vitro in cultured cancer cells. Since basal levels of lactate production were depleted through a lengthy glucose/pyruvate starvation process, the results can also accurately reflect the ratio of glycolysis versus pentose phosphate pathway, at least during the initial labeling phase (1-3 hours) (FIG. 6). It is known that the rate of glycolysis in cancer cells is affected by glucose uptake as well as several key glycolytic enzymes. Therefore, this method could be potentially used for assessing the efficacy of a variety of chemical compounds that target glucose uptake or different enzymes in the glycolysis process in cultured cancer cells. Likewise, this method can also be used for biomedical research detecting the effects of different genetic mutations on aerobic glycolysis in cancer cells, which may aid in the development of personalized therapy for cancer patients.

The majority of cancer-related deaths, including those in breast cancer, is caused by metastasis. Recent studies have shown that lactate can be used by adjacent cancer or stromal cells as an energy source to promote angiogenesis and metastasis (Doherty and Cleveland. 2013; Dhup et al., 2012). Indeed, the present results suggest that elevated lactate production from glycolysis is an indicator of tumor metastasis in breast cancer cell lines (Zhang et al., 2013; Wang et al., 2011). In fact, increased expression of multiple metastatic-related proteins has been reported in MDA-MB-231 rather than MDA-MB-453 cells or other low-or non-invasive breast cancer cell lines (Zhang et al., 2013; Wang et al., 2011), which is consistent with the present results. The results also agree a recent finding using isotopically labeled isogenic non-metastic versus metastatic cancer cells which show enhanced lactate production in metastatic cancer cells (Simoes et al., 2016).

Although mammography screenings have led to increased earlier detection of ductal carcinoma in situ or DCIS breast tumors (indolent abnormal cells confined within milk ducts), recent reports suggest that this method has failed to reduce breast cancer death from metastatic breast cancer because it cannot distinguish pre-invasive breast cancer from indolent breast cancer (Miller et al., 2014). While PET imaging technique using radiolabeled FDG is considered a method that mimics aerobic glycolysis rate in cancer cells, this method is not sensitive enough for detecting small lesions of breast tumors and cannot be used to detect pre-invasive cancer. Yet, the majority of the DCIS never become metastatic, and it is unclear why certain DCIS lesions develop into invasive breast cancer. As a result, a considerable number of patients suffer from aggressive treatment-related morbidities. Therefore, novel approaches and new technologies are urgently needed in searching for biomarkers suitable for detection of pre-invasive cancer.

Significant differences in incorporation rates of [1-¹³C]glucose into [3-¹³C]lactate were observed in serum samples obtained in mice with early stage metastatic mammary tumors or without tumors. Different from the in vitro cell study results, which measures glycolysis/PPP ratio, the relative rate of isotopic incorporation in vivo in the mouse study reflects the glycolysis rate using basal levels of lactate as the control. Therefore, it may be a better indicator for abnormal glycolysis from cancer cells in vim. It is conceivable that this approach could be further developed to measure elevated production of [3-¹³C]lactate in patients' serum samples as a biomarker for pre-invasive breast cancer, following the injection of a small amount of stable isotope-labeled [1-¹³C]glucose into patients after overnight fasting. This could be a minimally invasive, non-radioactive, and economic procedure that can be performed in women who have already developed DCIS breast tumors, detected by mammography screenings. The present results may thus pave the way for further exploration of the elevated production of stable isotopic lactate as a promising biomarker for pre-invasive breast cancer in clinical trials.

While several newly developed NMR-based techniques have been tested in their capability to detect invasive cancer, these techniques, like PET imaging, are much more expensive than the present technique and are still at early stage of development (Lupo et al., 2010; Pickup et al., 2008). In contrast, the present detection method could be a minimally invasive, non-radioactive, and economic procedure that can be performed in women who have already developed DCIS breast tumors, detected by mammography screenings. The present results thus may pave the way for further exploration of the elevated production of stable isotopic lactate as a promising biomarker for pre-invasive breast cancer in clinical trials.

In summary, the ability of cancer cells to produce large amounts of lactate through aerobic glycolysis (Warburg Effect) is considered one of the most consistent hallmarks of cancer, including breast cancer. It is known that elevated aerobic glycolysis is closely correlated with increased breast tumor aggressiveness and poor prognosis. Stable isotopic labeling is an effective method for determining the relative contribution of a substrate to a particular metabolic pathway when coupled to mass spectrometry (MS), which enables quantification of the relative abundance of molecules with different isotope composition. The sensitivity of liquid chromatography (LC)-MS technology makes it feasible to detect very low concentrations of small molecules or metabolites produced in cancer cells. Currently, there are no methods that can monitor the production of lactate from glucose through glycolysis in cancer cells without interference from other metabolic pathways. A positional isotopic labeling and LC-MS-based method was developed that can specifically measure the conversion of glucose to lactate through glycolysis in cancer cells. In addition, the rate of aerobic glycolysis obtained by this method was shown to be closely correlated with glucose uptake activity and lactate concentration in breast cancer cells. The results further demonstrate significantly elevated production of [3-¹³C]lactate in metastatic breast cancer cells and in early stage metastatic mammary tumors in mice, which may lead to the development of a promising biomarker for diagnosis and treatment of aggressive breast cancer.

### REFERENCES

Ben-Sahra et al., Science 339:1323 (2013).
Chambers et al., Nat. Biotechnol., 30:918 (2012).
Chen and Russo, Biochim Biophys. Acta, 1826:370 (2012).
Chen et al., Mol. Cancer Ther., 11:2212 (2012).
DeBerardinis et al., Cell Metab. 7: 11 (2008).
Dhup et al., Curr. Pharm. Des., 18:1319 (2012).
Doherty and Cleveland. J. Clin. Invest., 12:3685 (2012).
Fan et al., Mol. Cancer. 7:79 (2008).
Gaglio et al., Mol. Syst. Biol., 7:523 (2011).
Halaby et al., Cell Signal., 20:1555 (2009).
Hegeman, Brief Funct. Genomics, 9:139 (2010).
Lupo et al., Magn. Reson. Imaging. 21:153 (2010).
Miller et al., Bmj, 34:366 (2014).
Miller et al., Bmj, 348:g366 (2014).
Pickup et al., Magn. Reson. Med., 60:299 (2008).
Schupp et al., Magn. Reson. Med., 30:18 (1993).
Simoes et al., Neolasia. 12:671 (2016).
Smith et al., Anal. Chem., 718:779 (2006).
Ueda et al., Jpn. J. Clin. Oncol., 38:250 (2008).
Wang et al., Biochem Biophys. Res. Commun., 412:353 (2011).
Warburg, Science, 123:309 (1956).
Wolfender et al., J. Chromatol., 1382:136 (2015).
Zhang et al., Oncogene, 2:3375 (2014).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to detect aerobic glycolysis in a sample comprising cells, comprising:
a) providing a mixture comprising a sample obtained from mammalian cells and isotopically labelled glucose;
b) measuring in the mixture the conversion of isotopically labelled glucose to isotopically labelled lactate using liquid chromatography-mass spectrometry (LC-MS) or; and
c) determining glucose uptake, lactate concentration or the rate of aerobic glycolysis in the cells in the sample based on the presence or amount of the labelled lactate, the rate of conversion of the labelled glucose to the labelled lactate, in the mixture, or comparing the amount of converted lactate to the amount of converted lactate in corresponding cells.

2. The method of claim 1 wherein the label is ¹³C or deuterium (²H).

3. The method of claim 1 wherein the labelled glucose is [1-¹³C]glucose, [1,2-¹³C2]glucose, [¹³C6]glucose, or 6,6-deuterium labelled glucose.

4. The method of claim 1 wherein the presence of amount of [3-¹³C]lactate, the rate of conversion of [1-¹³C]glucose to [3-¹³C]lactate or the rate of conversion of deuterium labeled glucose to deuterium labelled lactate, is determined.

5-6. (canceled)

7. The method of claim 1 wherein the cells are breast cancer cells, prostate cancer cells, liver cancer cells, or ovarian cancer cells or have a selected genetic mutation.

8-11. (canceled)

12. The method of claim 1 wherein the glucose uptake, lactate concentration or the rate of aerobic glycolysis in the mixture is compared to a corresponding mixture having control cells or no cells.

13-32. (canceled)

33. A method to monitor cancer recurrence in a mammal, comprising:
a) providing a mixture comprising a sample from the mammal treated for cancer comprising cells and an amount of ¹³C or deuterium labelled glucose;
b) measuring in the mixture the conversion of the ¹³C or deuterium labelled glucose to ¹³C or deuterium labelled lactate using LC-MS; and
c) determining whether the mammal is at risk of recurrence based on the presence or amount of the ¹³C or deuterium labelled lactate, or the rate of conversion of the ¹³C or deuterium labelled glucose to ¹³C or deuterium labelled lactate, in the mixture.

34. The method of claim 33 wherein the mammal is a human treated for breast cancer.

35. The method of claim 33 wherein the presence or amount of [3-¹³C]lactate, or the rate of conversion of [1-¹³C]glucose to [3-¹³C]lactate, in the mixture is compared to the presence or amount of [3-¹³C]lactate, or the rate of conversion of [1-¹³C]glucose to [3-¹³C]lactate, in a control mixture or one or more samples from the mammal taken at an earlier point in time.

36. The method of claim 33 wherein the labelled glucose is [1-¹³C]glucose, [1,2-¹³C2]glucose, [¹³C6]glucose, or 6,6-deuterium labelled glucose.

37. The method of claim 33 wherein the sample is a physiological fluid sample.

38. A method to monitor a therapeutic response to a cancer therapy in a mammal having cancer, comprising:
a) providing a mixture comprising a sample from the mammal comprising cells and an amount of ¹³C or deuterium labelled glucose;
b) measuring in the mixture the conversion of the ¹³C or deuterium labelled glucose to ¹³C or deuterium labelled lactate using LC-MS; and
c) determining whether the mammal has a therapeutic response to the therapy based on the presence or amount of the ¹³C or deuterium labelled lactate, or the rate of conversion of the ¹³C or deuterium labelled glucose to ¹³C or deuterium labelled lactate, in the mixture.

39. The method of claim 38 wherein the mammal is a human.

40. The method of claim 38 wherein the presence or amount of [3-¹³C]lactate, or the rate of conversion of [1-¹³C]glucose to [3-¹³C]lactate, in the mixture is compared to the presence or amount of [3-¹³C]lactate, or the rate of conversion of [1-¹³C]glucose to [3-¹³C]lactate, in a control mixture or one or more samples from the mammal taken at an earlier point in time.

41. The method of claim 38 wherein the labelled glucose is [1-¹³C]glucose, [1,2-¹³C2]glucose, [¹³C6]glucose, or 6,6-deuterium labelled glucose.

42. The method of claim 38 wherein the sample is a physiological fluid sample.

43. The method of claim 38 wherein the sample is a physiological tissue sample.

44. The method of claim 42 wherein the sample is a blood sample, a urine sample or a milk sample.

45. The method of claim 38 wherein the mammal has breast cancer, prostate cancer, liver cancer or ovarian cancer.

46. The method of claim 38 wherein the ratio of [3-¹³C]lactate/unlabeled lactate or deuterium labeled lactate/unlabeled lactate is determined.
